(19) United States
(12) Patent Application Publication
(10) Pub. No.: US 2002/0025311 A1
(43) Pub. Date: Feb. 28, 2002

(54) Presbyopia treatment by lens alteration

(76) Inventor: Jonathan S. Till, Salem, VA (US)

Correspondence Address: KENYON & KENYON, 1500 K STREET, N.W., SUITE 700, WASHINGTON, DC, 20005, US

(21) Appl. No.: 09/930,287

(22) Filed: Aug. 16, 2001

Related U.S. Application Data
(60) Provisional application No. 60/225,659, filed on Aug. 16, 2000.

(51) Int. Cl. A61K 38/43; A61N 5/067; A61K 38/06; A61K 31/7105

(52) U.S. Cl. 424/94.1; 514/18; 514/47; 604/20

(57) ABSTRACT

This invention effects a change in the accommodation of the human lens affected by presbyopia through the use of various reducing agents that change accommodative abilities of the human lens, and/or by applying external energy to affect a change in the accommodative abilities of the human lens. By breaking bonds that adhere lens fibers together causing hardening of the lens, the present invention increases the elasticity and distensibility of the lens and/or lens capsule.

### BACKGROUND OF THE INVENTION

1. Field of the Invention

The present invention relates to a method and device for reversing and treating presbyopia.

2. Background of the Invention

Presbyopia affects virtually every person over the age of 44. According to Jobson Optical Database, 93% of people 45 and over are presbyopic. Presbyopia entails the progressive loss of amplitude of accommodation that occurs with aging. Adler's Physiology of the Eye, which is incorporated herein by reference, discloses that the human accommodative amplitude declines with age such that accommodation is substantially eliminated by the age of 50 to 55. Accommodative ability, as defined by U.S. Pat. No. 5,459,133 to Neufeld and incorporated in its entirety herein by reference, is the capacity of the eye to focus for near vision by changing the shape of the lens to become more convex.

The ocular tissues involved in the accommodative response include the lens, the zonules, the lens capsule, and the ciliary muscle. of these, the lens is the central tissue. These structures function together to enable the eye to focus on close objects by changing the shape of the lens. The lens is centrally suspended between the anterior and posterior chambers behind the pupillary opening of the iris. The lens is supported by a radially oriented array of zonules which extend from the lateral edges of the lens to the inner border of the circumferential ciliary muscle. The ciliary muscle is attached to the scleral coat of the eye. When the eye is at rest, it is focused for distance and the lens is in a somewhat flattened or less convex position. This shape is due to the tension that is exerted on the lens' periphery by the zonules. The zonules pull the edges of the lens toward the ciliary body.

During accommodation, the shape of the lens becomes more convex through contraction of the ciliary muscle which allows the ciliary attachment of the zonules to move toward the lens, reducing the tension in the zonules. This reduction in tension allows the central region of the lens to increase in convexity, thereby enabling near objects to be imaged on the retina. The processes involving the coordinated effort of the lens, zonules, ciliary body, medial rectus muscles and iris, among others, that leads to the ability of the eyes to clearly focus near on the retina is the accommodative process.

Several theories have been advanced to explain the loss of accommodation with age. These theories include the hardening of the lens with age; loss of strength in the ciliary muscle; and, the loss of elasticity of the lens capsule. As for the loss of strength of the ciliary muscle, it is noted that although there are age-related morphological changes that occur, there is little evidence of diminishing strength of the ciliary muscle. In fact, under the influence of pilocarpine, the ciliary muscle will vigorously contract even in presbyopic eyes.

As for changes in the lens capsule, it has been postulated that reduction in the elasticity of the capsule is, in fact, a contributing factor in presbyopia. Moreover, it has been found that Young's modules of elasticity for the lens capsule decreases by nearly 50% from youth to age 60, while accommodation decreases by 98%. Consequently, the principal cause of presbyopia is now considered to be “lenticular sclerosis” or the hardening of the lens. This hardening of the lens may be due to an alteration of the structural proteins or an increased adhesion between the lens fibers.

A cataract is a condition in which the lens becomes less clear. The study of cataracts lends insight into lens and capsular changes. The usual senile cataract is relatively discus-shaped when removed from the eye, its shape being dictated by the firm lens substance. The liquefied hypermature cataract is globular when extracted, rounded up by the elastic lens capsule. This is indirect evidence that it may be possible to reverse the lenticular changes associated with presbyopia, and that the lens capsule is still sufficiently elastic.

Other theories advanced to explain presbyopia involve the role of lens growth through life and the loss of tension on the lens capsule. These theories, however, have not been supported by clinical observations.

At the present time, common treatments for presbyopia include reading glasses, bifocal glasses, or mono-vision contact lenses. All of these solutions necessitate the use of an appliance creating additional shortcomings.

Alternative theories for treating presbyopia include scleral expansion and corneal reshaping. The efficacy of such techniques is not well-established and, importantly, these techniques to do not attempt to reverse what the inventors of the subject application believe to be a substantial causation, as explained more fully below, in the loss of the accommodative amplitude of the lens typically associated with the normal aging process. Thus, whereas the present invention as explained further below, is directed to a method of reversing or treating presbyopia resulting in underlying changes in the structures and/or interactions of molecules comprising those components of the eye associated with the accommodative process, most notably the lens and/or lens capsule, scleral expansion and corneal reshaping involve macroscopic changes in the morphology of the lens and cornea. Thus, the present invention provides a novel molecular approach to reversing presbyopia by restoring the accommodative amplitude of the lens, and in another preferred embodiment, to reversing presbyopia by such novel approach while also reducing the tendency for the lens to lose its thus restored accommodative amplitude.

Finally, the use of the excimer laser for the purposes of corneal reshaping to produce a multifocal refracting surface has been disclosed in U.S. Pat. No. 5,395,356. While this method seems promising, it still requires structural changes to compensate for aging.

### SUMMARY OF THE INVENTION

In its broadest sense, the present invention is directed to increasing the accommodative amplitude of the lens and thus to a method for reversing and/or treating presbyopia. In one embodiment, the present invention is directed to a method for reversing and/or treating presbyopia by breaking disulfide bonds in molecules comprising the structures of the eye, most notably the lens and the lens capsule, which disulfide bonds are believed to be a substantial factor in the progressive loss of accommodative amplitude. In another embodiment, the breaking of the disulfide bonds is accompanied by chemical modification of the free sulfur atoms formed upon breaking of the disulfide bonds, such chemical modification rendering the sulfur atoms less likely to form new disulfide bonds. This method thus comprises a method for preventing the recurrence of presbyopia by reducing the availability of new disulfide bonds to be formed. Particularly, this invention effects a change in the accommodative amplitude of the human lens by: (1) using various reducing agents that cause a change in the accommodative abilities of the human lens, and/or (2) the use of external energy to affect a change in the accommodative abilities of the human lens. It is believed that by breaking bonds, such as disulfides, that adhere lens fibers together and cause a hardening of the lens cortex, the present invention increases the elasticity and the distensibility of the lens cortex and/or the lens capsule.

Presbyopia, or the loss of the accommodative amplitude of the lens, has often advanced in a typical person age 45 or older to the point where some type of corrective lens in the form of reading glasses or other treatment is required. It is to be understood that loss of accommodative amplitude can occur in persons much younger or older than the age of 45, thus the present invention is not to be construed as limited to the treatment of presbyopia in a person of any particular age. The present invention is most useful in a person whose accommodative amplitude has lessened to a point where restoration thereof to some degree is desirable.

The accommodative amplitude of the lens is measured in diopters. The lens of a person who does not suffer from presbyopia (i.e. a person whose lens accommodates normally), will typically have an accommodative amplitude of about 2.5 diopters or greater. The terms “reversing presbyopia” or “treating presbyopia” as used in herein mean increasing the accommodative amplitude of the lens. The present invention is thus directed to a method for reversing presbyopia or increasing the accommodative amplitude of the lens of an individual. In a preferred embodiment of the present invention, the method of reversing presbyopia will result in an increase in the accommodative amplitude at least about by 0.5 diopters. In a more preferred embodiment of the present invention, the method of reversing presbyopia will result in an increase in the accommodative amplitude of at least about 2.0 diopters.

In an even more preferred embodiment, the method of reversing presbyopia of the present invention will result in an increase in the accommodative amplitude by at least about 5 diopters. In a most preferred embodiment of the present invention, the method of reversing presbyopia of the present invention will result in an increase of the accommodative amplitude of the lens to restoration thereof to that of a lens with a normal accommodative amplitude of 2.5 diopters or greater would result. While it is obviously most beneficial to restore the accommodative amplitude of the lens of each patient to a normal accommodative amplitude, it is to be understood that lesser degrees of restoration are also beneficial and in some cases due, for example, to a severe reduction in the accommodative amplitude (i.e. advanced presbyopia)it may not be possible to obtain full restoration to normal accommodative amplitude.

### DETAILED DESCRIPTION

As stated, inelasticity of the lens, or hardening thereof, is believed to be a contributing cause of presbyopia. The hardening of the lens may be due to an alteration of the structural proteins or an increased adhesion between the lens fibers. In one embodiment, the present invention is directed to treating presbyopia by altering the molecular and/or cellular bonds between the cortical lens fibers so as to free their movement with respect to each other. The increased elasticity of the lens apparatus can restore lost amplitude of accommodation. It is believed that disulfide bonds in the molecules comprising the structures of the eye responsible for proper accommodation are a substantial factor in the hardening of the lens and the concomitant loss of accommodative amplitude.

Thus, in one embodiment of the invention, a two-step process involves breaking the disulfide bond, and then protonating the newly-formed sulfur atom with a reducing agent such as glutathione to impart a hydrogen atom thereto. The steps can be performed simultaneously or consecutively. In either case, the reducing agent should be present at the time the disulfide bond is broken in order to eliminate reformation of disulfide. That is, the reducing agent introduces and bonds a moiety onto the free sulfur atom after breaking the disulfide bond such that the likelihood of reformation of another disulfide bond is prevented or at least reduced. While the reducing agent may introduce a hydrogen atom onto the free sulfur atom, thus forming a sulfahydryl (—SH group), the resultant —SH groups can again be oxidized to form a new disulfide bond. Thus, it is advantageous to introduce a group into the free sulfur atom, such a —CH₃ or other moiety, that reduces the tendency of new disulfide bond formation. This method can result in a substantial prevention of the reoccurrence of presbyopia.

As stated, it is believed that the disulfide bonds form between the lens fibers and substantially reduce the lens fibers' ability to easily move relative to each other and thus the ability of the lens to accommodate properly. While not wishing to be bound by any particular theory, the bonds may form by way of absorption of light energy, which causes the sulfahydryl bonds on the lens proteins to oxygenate removing a hydrogen atom from two adjacent —SH groups and creating water and a disulfide bond. Reducing the disulfide bonds requires hydrogen donors such as glutathione or other molecules.

The total refractive power of the lens is greater than what would be expected based on the curvature and the index of refraction. As stated, contraction of the ciliary muscle causes the ciliary body to move forward and towards the equator of the lens. This causes the zonules to relax their tension on the lens capsule, which allows the central lens to assume a more spherical shape. During accommodation, the main change is in the more central radius of curvature of the anterior lens surface, which is 12 mm in the unaccommodative state and can be 3 mm centrally during accommodation. Both the peripheral anterior and the posterior lens surfaces change very little in curvature during accommodation. The axial thickness increases while the diameter decreases. The central anterior lens capsule is thinner than the rest of the anterior capsule. This may explain why the lens bulges more centrally during accommodation. The thinnest portion of the capsule is the posterior capsule, which has a curvature greater than the anterior capsule in the unaccommodative state.

The protein content of the lens, almost 33% by weight, is higher than any other organ in the body. There are many chemical compounds of special interest in the lens. For example, glutathione is found in high concentration in the lens cortex even though there is very little in the aqueous. Thus, the lens has a great affinity for glutathione and actively absorbs, transports and synthesizes glutathione.

Approximately 93% of intralenicular glutathione is in the reduced form. Glutathione may be involved with maintaining the lens proteins, the sulfahydryl groups (—SH), in their reduced states. That is, after the disulfide bond is broken and the sulfur atoms are made available, glutathione can impart a hydrogen atom to form the sulfahydryl group thereby preventing or minimizing the reformation of a disulfide bond. In addition, ascorbic acid can also be found in very high concentrations in the lens. It is actively transported out of the aqueous and is at concentrations 15 times that found in the bloodstream. Both inositol and taurine are found at high concentrations in the lens for which the reason is not known.

According to one embodiment of the invention, the increase in the accommodative amplitude is accomplished by treatment of the outer lens region (the cortex) with radiation, heat, chemical, enzyme, gene therapy, nutrients, other energy source, and/or any combination of any of the above sufficient to break the disulfide bonds believed responsible for the inelasticity of the lens. Chemicals are useful to reduce disulfide bonds that are believed to anchor lens fibers hence preventing their free movement and elasticity. By making the anterior cortex more elastic, viscosity is lowered and the lens is again able to assume its characteristic central bulge during accommodation.

Chemicals suitable for causing reduction include, by way of example only, glutathione, ascorbic acid, Vitamin E, tetraethylthiuram disulfide, i.e., reducing agent, any biologically suitable easily oxidized compound, ophthalmic acid, inosotol, beta-carbolines, any biologically suitable reducing compound, reducing thiol derivatives with the structure:

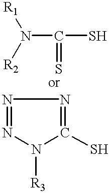

or disulfide derivatives with the structures:

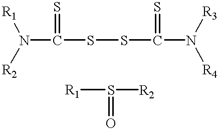

wherein R₁, R₂, R₃ and R₄ are independently a straight or branched lower alkyl which may be substituted, e.g., by hydroxyl, lower alkoxy or lower alkyl carbonyloxy, their derivatives or a pharmaceutically acceptable salt thereof. Preferred exemplary reducing agents include diethyl dithiocarbamate, 1-methyl-1H-tetrazol-5-yl-thiol and 1-(2-hydroxyethyl)-1H-tetrazol-5-yl-thiol or and pharmaceutically acceptable salts thereof. Other useful compounds can be found in U.S. Pat. No. 5,874,455 which is hereby incorporated in its entirety by reference. The above-listed chemicals are merely exemplary and other reducing agents which behave similarly by breaking the disulfide bond are included within the scope of this invention.

The chemical reducing agents can be used alone or in conjunction with a catalyst such as an enzyme. Enzymes and other nutrients suitable for causing or facilitating reduction include, for example, aldoreductase, glyoxylase, glutathione S-transferase, thiol reductase, tyrosine reductase or any compatible reductase. Again, it should be noted that the above-listed enzymes are exemplary and not an exhaustive list. The enzymes can be naturally present in the eye, or can be added to the eye together with or separate from the chemical reducing agent or energetic means disclosed herein. As such, other chemically and biologically comparable enzymes which help break disulfide bonds or behave similarly should be considered as within the scope of the present invention.

In one embodiment of the invention, the reduction of disulfide groups of the lens proteins to sulfahydryl groups is accomplished by delivering to the lens a compound such as glutathione, thiols, or others in sufficient quantities to reduce the disulfide bonds and other molecular and cellular adhesions. Other enzymes or chemicals that affect a methylation on the free sulfur atom include for example, methyl-methane tiosulfonate, methyl glutatione, S-methyl glutatione, S-transferase and other biologically compatible methylating agent. Use of emulsions such as nanocapsules, albumin microspheres, carrier molecules such as inositol, taurine or other biologically suitable means for delivering the reducing agent to the lens is an integral part of this invention. The chemical reducing agent will typically be delivered in the form of a solution or suspension in an opthalmically acceptable carrier. In some cases, the application of external energy to affect or catalyze the reduction of the disulfide bonds as well as the disruption of other bonds and adhesions, may be beneficial. The application of external energy alone can be used to break the disulfide bonds. Externally applied energy can have any form, by way of example only, any of laser, ultrasound, heat, ionizing, light, magnetic, microwave, sound, electrical, or other not specifically mentioned, can be used alone or in combination with the reducing agents to affect the treatment of presbyopia, or a combination of any of these types of energy.

In a similar manner, agents can be delivered to the lens capsule which bind or interact with the capsule to affect greater elasticity or distensibility. Such agents either cause the capsule to shrink in surface area or increase the tension of the lens capsule on the peripheral anterior or posterior of the lens. Externally applied energy can have any form, by way of example only, any of laser, ultrasound, heat, ionizing, light, magnetic, microwave, sound, electrical, or other not specifically mentioned can be used alone or in combination with the reducing agents to affect the treatment of presbyopia or a combination of any of these types of energy.

In another embodiment of the invention, externally applied energy can be used as a catalyst to induce or increase the rate of the reduction reaction. Thus, by applying external energy, the peripheral portion of the capsule is preferentially affected, leaving the central 4 mm zone of accommodation unaffected. This allows the lens to assume a more accommodative state. The externally applied energy can also be applied alone to promote the reduction reaction and the cellular changes that ultimately affect the lens′ cortex.

As examples, lasers useful in the present invention include: excimer, argon ion, krypton ion, carbon dioxide, helium-neon, helium-cadmium, xenon, nitrous oxide, iodine, holmium, yttrium lithium, dye, chemical, neodymium, erbium, ruby, titanium-sapphire, diode, any harmonically oscillating laser, or any other electromagnetic radiation. Exemplary forms of heating radiation include: infrared, heating, infrared laser, radiotherapy, or any other methods of heating the lens. Finally, exemplary forms of sound energy that can be used in an embodiment of the invention include: ultrasound, any audible and non-audible sound treatment, and any other biologically compatible sound energy.

The external energy used with various embodiments and methods of the present invention could be applied through either contact with the sclera or cornea, non-contact techniques, or through intraocular methods of delivery. More than one treatment may be needed to effect a suitable increase in the accommodative amplitude. When more than one modality of treatment is desirable, chemical treatment can be administered prior to, after, or simultaneously with the application of energy.

In an exemplary embodiment, a treatment can comprise administering a composition of one or more active agents suspended in biocompatible carrier. In another exemplary embodiment, the active agents can be administered in a solution or suspension containing ophthalmically acceptable sterile viral phage. The phage can be introduced to the lens by, for example, topical eye drop or administered systematically a pill or as an injection into either the blood stream or the lens itself. The carrier can include, for example, balanced salt solution or saline. The active agents can include thiol transferase in an amount of 0 to 20% by volume, preferably 2 to 10% by volume, glutathione in an amount of 0 to 20% by volume, with a preferred range of 2 to 10% by volume, and nicotinamide adenine dinucleotide phosphate (NADP) in an amount of 0-20% by volume, with a preferred range of 2-10% by volume. The balance can comprised of a biocompatible carrier. The composition can be administered in total drop volumes of 0.1 to 2.5 ml with a referred range of 0.25 to 1 ml.

In another embodiment, thiol transferase can be altered to become photo reactive. Upon administering the composition having thiol transferase (2-10% by Vol.), glutathione (2-10% by vol.) and NADP (2-10%), a focused energy source such as laser can be applied to activate thiol transferase and the subsequent reduction of the disulfide bonds.

What is claimed is:

1. A method for reversing presbyopia comprising applying localized energy to the area to be treated and administering a pharmaceutically sufficient quantity of a biologically acceptable chemical substance capable of breaking the chemical bonds between disulfates of the cortical lens fibers.

2. The method of claim 1, wherein said localized energy comprises treatment with at least one or more of heat, energy, sound or enzyme.

3. The method of claim 1, wherein said biologically acceptable chemical comprises glutathione, thiols and derivatives thereof.

4. A method for increasing the amplitude of accommodation of a human eye having a lens and a ciliary muscle comprising the step of administering a pharmaceutically sufficient quantity of a biologically acceptable reducing agent to affect a change in the elasticity of the human lens.

5. The method of claim 4, wherein the biologically acceptable reducing agent is selected from the group consisting of glutathione, thiols and derivatives thereof.

6. The method of claim 4, further comprising the step of treating the human eye with external energy.

7. The method of claim 1, wherein reformation of disulfide bonds is prevented.

8. A method for treating presbyopia comprising breaking disulfide bonds formed about the lens fibers to form sulfides and reducing them with either hydrogen or other agents.

9. The method of claim 8, further comprising catalyzing the reaction by applying energy.

10. The method of claim 8, wherein said disulfide bond breaking is catalyzed by agents selected from the group consisting of aldoreductase, glyoxylase, glutathione S-transferase, thiol reductase, tyrosine reductase or any biologically suitable compatible reductase.

11. A method for treating presbyopia comprising breaking disulfide bonds and reforming the sulfide bonds with —CH3 or any other suitable molecule.

12. The method of claim 11, wherein said breaking disulfide bonds further comprises the applying external energy.

13. The method of claim 11, wherein said breaking disulfide bonds further comprises applying enzyme capable of breaking the disulfide bonds.

14. The method of claim 13, wherein said enzyme comprises S-methyl glutatione, S-Transferase.

15. The method of claim 11, wherein said breaking disulfide bonds further comprises applying a chemical catalyst capable of promoting a catalytic reaction.

16. The method of claim 15, wherein said chemical catalyst comprises methyl-methane thiosulfonate and methyl glutatione.

17. A method for treating presbyopia comprising breaking interlenticular fiber adhesions and freeing the fibers to move relative to each other.

18. The method of claim 17, wherein said breaking interlenticular fiber adhesions further comprises applying external energy.

19. The method of claim 17, wherein said breaking interlenticular fiber adhesions further comprise applying enzyme capable of breaking said interlenticular fiber adhesions.

20. The method of claim 17, wherein said breaking interlenticular fiber adhesions further comprise applying a chemical catalyst capable of promoting a catalytic reaction.

21. A method for reversing presbyopia comprising applying localized energy to the area to be treated and administering a pharmaceutically sufficient quantity of a biologically acceptable chemical substance capable of breaking the chemical bonds between disulfates of the cortical lens fibers.

22. An agent that prevents or reduces the likelihood of reformation of disulfide bonds.

23. A pharmaceutical composition for treatment of presbyopia comprising thiol transferase, glutatione, nicotineamid adenine dinucleotide phosphate.

24. The pharmaceutical composition of claim 23, further comprising a biocompatible carrier.

25. The pharmaceutical composition of claim 23 encased in a viral phage.

26. The pharmaceutical composition of claim 24, wherein the composition is administered topically.

27. The pharmaceutical composition of claim 23 administered systematically.

28. The composition of claim 23, further comprising a photo reactive compound.

29. The composition of claim 28, wherein the composition is activated by introduction of external energy.

30. The composition of claim 23, wherein the thiol transferase is present in an amount of 0-20% by volume.

31. The composition of claim 23, wherein the glutatione is present in an amount of 0-20% by volume.

32. The composition of claim 23, wherein nicotineamid adenine dinucleotide phosphate is present in an amount of 0-20% by volume.

33. The composition of claim 23, wherein the glutatione is S-glutathione.
